(19) United States
(12) Patent Application Publication
(10) Pub. No.: US 2014/0336218 A1
(43) Pub. Date: Nov. 13, 2014

(54) ARFORMOTEROL AND TIOTROPIUM COMPOSITIONS AND METHODS FOR USE

(71) Applicant: SUNOVION PHARMACEUTICALS INC., Marlborough, MA (US)

(72) Inventors: Holly HUANG, Northborough, MA (US); Elizabeth B. GOODWIN, Upton, MA (US); Kendyl M. SCHAEFER, Dedham, MA (US); John P. HANRAHAN, West Roxbury, MA (US); William T. ANDREWS, Hopkinton, MA (US); Paul MCGLYNN, Northborough, MA (US)

(73) Assignee: SUNOVION PHARMACEUTICALS INC., Marlborough, MA (US)

(21) Appl. No.: 14/340,736

(22) Filed: Jul. 25, 2014

Related U.S. Application Data
(63) Continuation of application No. 13/125,506, filed on Jun. 14, 2011.
(60) Provisional application No. 61/107,964, filed on Oct. 23, 2008.

(51) Int. Cl. A61K 31/46; A61K 9/00; A61K 31/137

(52) U.S. Cl. CPC A61K 31/46; A61K 31/137; A61K 9/0078
USPC 514/304

(57) ABSTRACT

Compositions and methods for the prevention and/or treatment of airway and/or respiratory disorders are provided. The compositions comprise arformoterol (the (R,R)-formoterol isomer) and tiotropium.

### CROSS REFERENCE TO RELATED APPLICATION

This application claims priority to U.S. Provisional Application No. 61/107,964 filed Oct. 23, 2008, the entire disclosure of which is herein incorporated by reference.

### FIELD OF THE INVENTION

The present inventions relate to compositions comprising arformoterol (the (R,R)-formoterol isomer) and tiotropium for the prevention and/or treatment of airway and/or respiratory disorders. In various embodiments the compositions are suitable for use in a nebulizer.

### BACKGROUND OF THE INVENTION

Asthma, bronchitis and emphysema are known as Chronic Obstructive Pulmonary Diseases (COPD). COPD is characterized as generalized airways obstruction, particularly of small airways, associated with varying degrees of symptoms of chronic bronchitis, asthma, and emphysema. Worldwide, COPD is one of the most prevalent noninfectious diseases in the world. The health and cost burden of COPD is even more substantial as it contributes to other serious co-morbidities including osteoporosis, fractures, respiratory infections, lung cancer, and cardiovascular disease.

The term COPD was introduced because these conditions often coexist, and it may be difficult in an individual case to decide which is the major condition producing the obstruction. Airways obstruction is defined as an increased resistance to airflow during forced expiration. It may result from narrowing or obliteration of airways secondary to intrinsic airways disease, from excessive collapse of airways during a forced expiration secondary to pulmonary emphysema, from bronchospasm as in asthma, or may be due to a combination of these factors. Although obstruction of large airways may occur in all these disorders, particularly in asthma, patients with severe COPD characteristically have major abnormalities in their small airways, namely those less than 2 mm internal diameter, and much of their airways obstruction is situated in this zone. The airways obstruction is irreversible except for that which can be ascribed to asthma.

Asthma is a reversible obstructive lung disorder characterized by increased responsiveness of the airways. Asthma can occur secondarily to a variety of stimuli. The underlying mechanisms are unknown, but inherited or acquired imbalance of adrenergic and cholinergic control of the airways diameter has been implicated. Persons manifesting such imbalance have hyperactive bronchi and, even without symptoms, bronchoconstriction may be present. Overt asthma attacks may occur when such persons are subjected to various stresses, such as viral respiratory infection, exercise, emotional upset, nonspecific factors (e.g., changes in barometric pressure or temperature), inhalation of cold air or irritants (e.g., gasoline fumes, fresh paint and noxious odors, or cigarette smoke), exposure to specific allergens, and ingestion of aspirin or sulfites in sensitive individuals. Psychologic factors may aggravate an asthmatic attack but are not assigned a primary etiologic role.

Persons whose asthma is precipitated by allergens (most commonly airborne pollens and molds, house dust, animal danders) and whose symptoms are IgE-mediated are said to have allergic or “extrinsic” asthma. They account for about 10 to 20% of adult asthmatics; in another 30 to 50%, symptomatic episodes seem to be triggered by non-allergenic factors (e.g., infection, irritants, emotional factors), and these patients are said to have nonallergic or “intrinsic” asthma. In many persons, both allergenic and nonallergenic factors are significant. Allergy is said to be a more important factor in children than in adults, but the evidence is inconclusive.

Chronic bronchitis (unqualified) is a condition associated with prolonged exposure to nonspecified bronchial irritants and accompanied by mucus hypersecretion and certain structural changes in the bronchi. Usually associated with cigarette smoking, it is characterized clinically by chronic productive cough. The term chronic obstructive bronchitis is used when chronic bronchitis is associated with extensive abnormalities of the small airways leading to clinically significant airways obstruction. (Pulmonary emphysema is enlargement of the air spaces distal to terminal nonrespiratory bronchioles, accompanied by destructive changes of the alveolar walls.) The term chronic obstructive emphysema is used when airways obstruction is also present and where it is clear that the major features of the disease can be explained by emphysematous changes in the lungs.

There is a need for compositions and methods for the prevention and/or treatment of airway and/or respiratory disorders.

### SUMMARY OF THE INVENTION

The present invention relates to compositions comprising arformoterol (the (R,R)-formoterol isomer) and tiotropium for the prevention and/or treatment of airway and/or respiratory disorders. In various embodiments, provided are arformoterol and tiotropium compositions suitable for use in a nebulizer.

In various embodiments, the composition comprises a liquid for nebulization comprising arformoterol and tiotropium, wherein the composition is substantially free of the (S,S), (R,S) and (S,R) stereoisomers of formoterol. In various embodiments, the formoterol component of said compositions comprises greater than about 99% by weight arformoterol and less than about 1% by weight of the other stereoisomers of formoterol.

In some aspects, the present invention relates to a pharmaceutical composition comprising tiotropium, or a pharmaceutically acceptable salt thereof, and arformoterol, or a pharmaceutically acceptable salt thereof, together in water or a water-ethanol mixture.

In other aspects, the present invention relates to a liquid, propellant-free pharmaceutical composition comprising (a) tiotropium, or a pharmaceutically acceptable salt, hydrate or solvate thereof, in an amount between about 5 μg to about 30 μg based on tiotropium; and (b) a formoterol component comprising arformoterol, or a pharmaceutically acceptable salt, hydrate or solvate thereof, in an amount between about 6 μg to about 40 μg based on arformoterol; wherein the tiotropium and formoterol component are dissolved together in a liquid carrier, and wherein the formoterol component comprises less than about 10% by weight of stereoisomers of formoterol other than arformoterol.

In some aspects, the present invention relates to a medicament comprising to a liquid, propellant-free pharmaceutical composition comprising (a) tiotropium, or a pharmaceutically acceptable salt, hydrate or solvate thereof, in an amount between about 5 μg to about 30 μg based on tiotropium; and (b) a formoterol component comprising arformoterol, or a pharmaceutically acceptable salt, hydrate or solvate thereof, in an amount between about 6 μg to about 40 μg based on arformoterol; wherein the tiotropium and formoterol component are dissolved together in a liquid carrier, and wherein the formoterol component comprises less than about 10% by weight of stereoisomers of formoterol other than arformoterol, wherein the medicament is provided in an ampoule as a liquid for nebulization.

In others aspects, the present invention relates to a method of treating conditions associated with reversible obstruction of the airways comprising the administration of a liquid, propellant-free pharmaceutical composition comprising (a) tiotropium, or a pharmaceutically acceptable salt, hydrate or solvate thereof, in an amount between about 5 μg to about 30 μg based on tiotropium; and (b) a formoterol component comprising arformoterol, or a pharmaceutically acceptable salt, hydrate or solvate thereof, in an amount between about 6 μg to about 40 μg based on arformoterol; wherein the tiotropium and formoterol component are dissolved together in a liquid carrier, and wherein the formoterol component comprises less than about 10% by weight of stereoisomers of formoterol other than arformoterol, wherein the method comprises administering a total per day dose of arformoterol between about 6 to about 150 μg and a total per day dose of tiotropium between about 8 to about 150 μg.

### BRIEF DESCRIPTION OF THE FIGURES

FIG. 1: Patient disposition for study of Example 1.

FIG. 2A: Data from the study of Example 1 showing mean change in FEV₁ from study baseline at week 2.

FIG. 2B: Data from the study of Example 1 showing mean change in time normalized FEV₁AUC₀₋₂₄ from study baseline at week 2.

FIG. 3: Data from the study of Example 1 showing change in inspiratory capacity from study baseline at week.

### DETAILED DESCRIPTION OF THE INVENTION

Nebulizers provide a means of administering drugs to the airways of a patient whilst the patient breathes at an approximately normal rate. They can be particularly suitable for patients who are unable, whether due to age or injury or otherwise, to inhale at the much higher rates often required for administration of drugs via metered dose inhalers or dry powder inhalers and for patients who cannot for whatever reason coordinate the activation of the metered dose inhaler with their inhalation of breath. A nebulizer apparatus creates a vapor containing drug and the patient breathes the vapor via a mouthpiece or mask attached to the nebulizer. Typically, nebulizers are used to deliver drugs for the treatment of airways disorders such as asthma and COPD. Accordingly, in various embodiments the present invention provides novel nebulizer compositions, suitable for treatment of COPD, asthma and/or other conditions associated with reversible obstruction of the airways.

In various aspects, the present inventions provide methods of treatment of COPD, asthma and/or other conditions associated with reversible obstruction of the airways comprising administering, via a nebulizer, a composition comprising both arformoterol and tiotropium in a pharmaceutically acceptable carrier.

### Definitions

The term “formoterol component” as used herein means the total of all stereoisomers of formoterol in a composition of the present inventions.

The term “substantially free of other stereoisomers of formoterol ” as used herein means that the total formoterol component of a composition of the present inventions contains less than about 10% by weight of formoterol stereoisomers other than (R,R) formoterol. In various preferred embodiments, the formoterol component of a composition of the present inventions contains at least 99% by weight of (R,R) formoterol and 1% or less of other stereoisomers of formoterol.

The term “eliciting a bronchodilator effect” means relief from the symptoms associated with obstructive airway diseases, which include but are not limited to respiratory distress, wheezing, coughing, shortness of breath, tightness or pressure in the chest and the like.

The phrase “therapeutically effective amount” as used herein means that amount of a compound, material, or composition comprising a compound of the present invention which is effective for producing some desired therapeutic bronchodilator effect at a reasonable benefit/risk ratio applicable to any medical treatment.

The term “pharmaceutically acceptable salts” includes, but is not limited to, salts of the active compounds which are prepared with relatively nontoxic acids or bases, depending on the particular substituents found on the compounds described herein. It is to be understood that the various salts can also include hydrates thereof.

When an active ingredient of a composition of the present inventions contains relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt.

When an active ingredient of a composition of the present inventions contains relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

Also included are salts of amino acids such as arginate and the like, and salts of organic acids like glucuronic or galactunoric acids and the like (see, for example, Berge et al., Journal of Pharmaceutical Science, 66: 1-19 (1977)).

### Arformoterol & Tiotropium

Formoterol, whose chemical name is (+/−) N-[2-hydroxy-5-[1-hydroxy-2[[2-(p-methoxyphenyl)-2-propyl]amino]ethyl]phenyl]-formamide, is a highly potent and β₂-selective adrenoceptor agonist having a long lasting bronchodilating effect when inhaled. Formoterol has two chiral centers in the molecule, each of which can exist in two possible configurations. This gives rise to four combinations: (R,R), (S,S), (R,S) and (S,R). (R,R) and (S,S) are mirror images of each other and are therefore enantiomers; (R,S) and (S,R) are similarly an enantiomeric pair. The mirror images of (R,R) and (S,S) are not, however, superimposable on (R,S) and (S,R), which are diastereomers. Arformoterol is the (R,R) stereoisomer of formoterol.

In various embodiments, the compositions comprise (R,R)-formoterol L-(+)-tartrate, predominantly in the polymorphic form A, as described in U.S. Pat. No. 6,268,533, the entire contents of which are herein incorporated by reference.

Tiotropium, whose chemical name is (1α, 2β, 4β, 5α, 7β-7-[(Hydroxydi-2-thienylacetyl)oxy]-9,9-dimethyl-3-oxa-9-azoniatricyclo[3.3.1.0 2,4]nonane, is a muscarinic receptor antagonist, and acts as a long-acting anticholinergic brochodilator. Tiotropium, is the free ammonium cation, and tiotropium in the form of a salt typically contains an anion as counter-ion.

### Pharmaceutical Compositions

The pharmaceutical compositions of the present invention comprise (R,R) formoterol and tiotropium as active ingredients. The active ingredients can be present as a pharmaceutically acceptable salt, hydrate or solvate thereof. The compositions can also contain one or more pharmaceutically acceptable carriers and additives. The term “pharmaceutically acceptable carrier and additives” includes, but is not limited to, vehicles, propellants, diluents, excipients, complexing agents, stabilizers, granulating agents, lubricants, binders, disintegrating agents, cosolvents, adjuvants, additives and other elements appropriate for incorporation into a pharmaceutical composition. The carrier(s) and additive(s) are “acceptable” in the sense of being compatible with the other ingredients of the composition and not deleterious to the recipient thereof.

In various embodiments, suitable pharmaceutically acceptable salts for the formoterol component include acetate, benzenesulfonate (besylate), benzoate, camphorsulfonate, citrate, ethenesulfonate, fumarate, gluconate, glutamate, hydrobromate, hydrochlorate, isethionate, lactate, maleate, malate, mandelate, methanesulfonate, salt of mucic acid, nitrate, pamoate, salt of pantothenate acid, phosphate, succinate, salts of sulfuric acid, tartrate, p-toluenesulfonate, and the like. In various embodiments, the fumaric acid salt (fumarate) is preferred. In various embodiments the tartrate salt is preferred.

In various embodiments, suitable pharmaceutically acceptable salts for the tiotropium component include salts where the counter-ion comprises chloride, bromide, iodide, methanesulfonate, p-toluenesulfonate, and/or methylsulfate. In various embodiments, tiotropium bromide monohydrate is preferred.

The compositions of the present inventions include compositions such as suspensions, solutions, aerosols (e.g., hydrofluoralkane (HFA aerosols)). The most preferred route for administration of the compositions of the present inventions is by inhalation. Administration by inhalation includes, but is not limited to, administration by inhalation powder, inhalation aerosol and inhalation solution. Various examples of methods of administration include, but are not limited to, by dry powder inhaler (DPI), by metered-dose inhaler (MDI) and by nebulizer.

In various embodiments comprising a liquid for nebulization (e.g., an inhalation solution composition), the carrier is preferably water or water-ethanol and may comprise other components. A pharmaceutically acceptable carrier is preferably buffered for human use to a pH of about 3.0 to about 5.5.

One or more tonicity adjusting agents can be added to provide the desired ionic strength of an inhalation solution. Tonicity adjusting agents for use herein include, but are not limited to, those which display no or only negligible pharmacological activity after administration. Both inorganic and organic tonicity adjusting agents can be used. Compositions of the inventions can also include excipients and/or additives. Examples of these include, but are not limited to, surfactants, stabilizers, complexing agents, antioxidants, or preservatives which prolong the duration of use of the finished pharmaceutical composition, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In various embodiments, the complexing agent is EDTA. Antioxidants include, but are not limited to, vitamins, provitamins, ascorbic acid, vitamin E or salts or esters thereof. Preservatives include, but are not limited to, those that protect the solution from contamination with pathogenic particles, including, for example, benzalkonium chloride or benzoic acid, or benzoates such as sodium benzoate. In various embodiments, the compositions are free of preservative, which is an advantage as some preservatives can be associated with bronchoconstrictor effects the opposite effect to that required by the composition.

In various embodiments, the compositions comprise tiotropium and arformoterol in water or a water-ethanol mixture, or a pharmaceutically acceptable salt, hydrate or solvate of these active ingredients.

In various embodiments, the compositions comprise a liquid, propellant-free pharmaceutical composition comprising: (a) tiotropium, or a pharmaceutically acceptable salt, hydrate or solvate thereof, in an amount between about 5 μg to about 30 μg based on tiotropium; and (b) a formoterol component comprising arformoterol, or a pharmaceutically acceptable salt, hydrate or solvate thereof, in an amount between about 6 μg to about 40 μg based on arformoterol; in a (c) a carrier selected from water or a water/ethanol mixture; wherein the active ingredients are dissolved in the carrier; wherein the liquid composition has a pH in the range between about 3.0 to about 5.5; and wherein the formoterol component comprises less than about 10% by weight of stereoisomers of formoterol other than arformoterol. In various embodiments, the pH of the liquid composition is between about 3 to about 4. In various embodiments, the formoterol component comprises greater than about 99% by weight of arformoterol and less than about 1% by weight of stereoisomers of formoterol other than arformoterol. In various embodiments, a liquid, propellant-free pharmaceutical composition is provided with a total liquid volume between about 1 ml to about 3 ml. In various embodiments, the liquid, propellant-free pharmaceutical composition is provided with a total liquid volume of less than 2 ml. In various embodiments, the tiotropium, or a pharmaceutically acceptable salt, hydrate or solvate thereof, is present in an amount between about 5 μg to about 15 μg based on tiotropium; and the formoterol component comprising arformoterol, or a pharmaceutically acceptable salt, hydrate or solvate thereof, is present in an amount between about 6 μg to about 30 μg based on arformoterol.

It is to be understood that herein, the amount of active ingredient (e.g., tiotropium and/or arformoterol) refers to the weight of active ingredient itself and does not include the weight of any salt, water, etc. of the salt, hydrate etc. of the compound. For example to provide 15 μg of arformoterol (based on arformoterol) from an arformoterol tartrate salt, would require about 22 μg of the arformoterol tartrate salt. Similarly, to provide 18 μg of tiotropium (based on tiotropium) from tiotropium bromide monohydrate, would require about 22.5 μg of the tiotropium bromide monohydrate.

Pharmaceutical compositions of the present inventions containing arformoterol and tiotropium can be presented, for example, in unit dosage form (e.g., in an ampoule as a liquid for nebulization), and in multiple dosage forms (e.g., as a metered dose inhaler). Preferred dosages are those containing an effective combined dose, or an appropriate fraction thereof, of the active ingredients, or a pharmaceutically acceptable salt, hydrate or solvate thereof. The magnitude of a prophylactic or therapeutic dose typically varies with the nature and severity of the condition to be treated and the route of administration. The dose, and perhaps the dose frequency, will also vary according to the age, body weight and response of the individual patient. Further, it is noted that the clinician or treating physician knows how and when to interrupt, adjust or terminate therapy in conjunction with individual patient's response.

In various preferred embodiments, the dosage amounts and methods of treatment associated therewith, comprise once per day or twice per day administration of a composition of the present inventions. In various embodiments, the per dose amount is such that the total per day dose of arformoterol is between about 6 to about 150 μg (preferably 15-45 μg) and the total per day dose of tiotropium is about 8 to about 150 μg (preferably 18-54 μg).

In various aspects, the present inventions provide methods of treatment of COPD, asthma and/or other conditions associated with reversible obstruction of the airways comprising administering, via a nebulizer, a composition comprising both arformoterol and tiotropium in a pharmaceutically acceptable carrier.

In various aspects the present inventions provide methods for preventing bronchoconstriction or inducing bronchodilation in a mammal by administering a composition comprising: (a) tiotropium, or a pharmaceutically acceptable salt, hydrate or solvate thereof, in an amount between about 5 μg to about 30 μg based on tiotropium; and (b) a formoterol component comprising arformoterol, or a pharmaceutically acceptable salt, hydrate or solvate thereof, in an amount between about 6 μg to about 40 μg based on arformoterol; in a (c) a carrier selected from water or a water/ethanol mixture; wherein the active ingredients are dissolved in the carrier; wherein the liquid composition has a pH in the range between about 3.0 to about 5.5; and wherein the formoterol component comprises less than about 10% by weight of stereoisomers of formoterol other than arformoterol. In various embodiments, the pH of the liquid composition is between about 3 to about 4. In various embodiments, the formoterol component comprises greater than about 99% by weight of arformoterol and less than about 1% by weight of stereoisomers of formoterol other than arformoterol. In various embodiments, a liquid, propellant-free pharmaceutical composition is provided with a total liquid volume between about 1 ml to about 3 ml. In various embodiments, the liquid, propellant-free pharmaceutical composition is provided with a total liquid volume of less than 2 ml. In various embodiments, the tiotropium, or a pharmaceutically acceptable salt, hydrate or solvate thereof, is present in an amount between about 5 μg to about 15 μg based on tiotropium; and the formoterol component comprising arformoterol, or a pharmaceutically acceptable salt, hydrate or solvate thereof, is present in an amount between about 6 μg to about 30 μg based on arformoterol.

Various aspects of the present inventions may be further understood in light of the following further examples, which are not exhaustive and which should not be construed as limiting the scope of the present teachings in any way.

### FURTHER EXAMPLES

### Example 1

### Clinical Trial: COPD Patients

### Summary of Trial

A randomized double-blind study was conducted and compared pulmonary function and symptom improvement among patients treated with arformoterol mono-therapy, tiotropium mono-therapy, and both therapies combined, and tested the hypothesis that the combined therapy would afford significantly greater efficacy than either single-therapy.

This was a 2-week, prospective, multi-center (34 sites), randomized, modified blind, double dummy, parallel group study designed to evaluate the efficacy and safety of the combination of arformoterol 15 μg BID and tiotropium 18 μg QD (dosed sequentially) versus the individual mono-therapies in the treatment of COPD patients. The study was conducted according to the principles established by the Declaration of Helsinki (see, e.g., World Medical Association Declaration of Helsinki. Recommendations guiding physicians in biomedical research involving human subjects. JAMA 1997; 277:925-926.). Appropriate Institutional Review boards approved the protocol and written informed consent was obtained from the patients.

### Study Patients

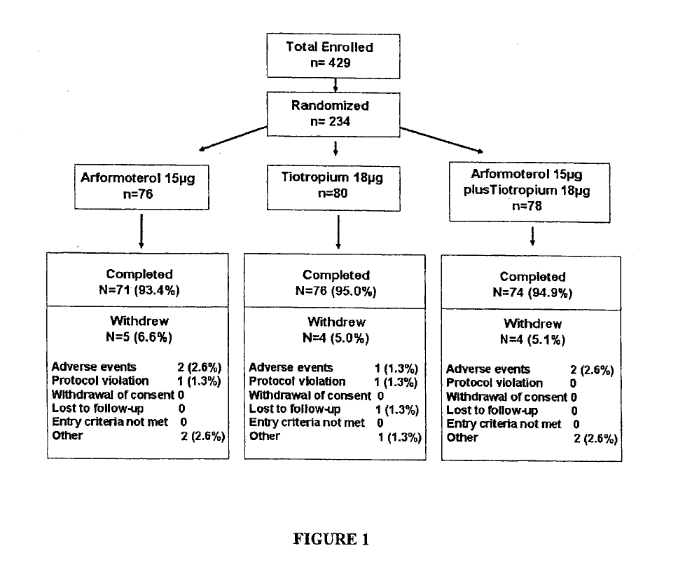

Of 429 patients screened, 235 were randomized to treatment and 234 received at least one dose of study medication (intent-to-treat population, [ITT]) (See FIG. 1). All patients had non-asthmatic COPD (including emphysema and/or chronic bronchitis). Eligible patients were at least 45 years of age had a ≧15 pack-year history of smoking, and had a breathlessness severity based on Medical Research Council Dyspnea Score (34)≧2. They also were required to have a pre-bronchodilator baseline pulmonary function of FEV₁>0.7 L, FEV₁/FVC ratio of ≦70%, and FEV₁≦65% predicted. Patients were excluded if they had life-threatening or unstable respiratory status within 30 days of the screening visit. Patients who changed their prescribed dose or type of COPD medication within 14 days prior to screening or who had ever used tiotropium bromide inhalation powder were excluded.

During the study period, the use of LABAs or long-or short-acting anticholinergic bronchodilators (except for the study medication) was prohibited. Use of oral and inhaled corticosteroids was allowed as long as patients were on a stable dosing regimen for at least 14 days prior to study entry that was maintained throughout the study. Patients were required to withhold oral corticosteroids for at least 24 hours prior to pulmonary function testing. Leukotriene modifiers and methylxanthines were not allowed for at least 7-days prior to study entry. Levalbuterol MDI (Xopenex® Sepracor Inc., Marlborough, Mass.) was supplied and used as-needed for rescue medications for acute bronchospasm and acute treatment of COPD symptoms throughout the trial. Patients were instructed to withhold the use of rescue medication for ≧6 hours prior to each clinic visit.

### Study Protocol

At the screening visit, baseline values were obtained for COPD symptoms, Modified Medical Research Council (MMRC) Dyspnea Scale, heart rate, vital signs, and pulmonary function tests. Medical event calendars and medication logs that were to be completed daily, and rescue medication were also dispensed. The medication logs were used to assess compliance by monitoring the number of UDV/DPI doses taken.

Eligible patients were randomized to receive one of three treatments for 14 days: nebulized arformoterol 15 μg (Brovana®, Sepracor Inc., Marlborough, Mass.) BID and placebo DPI QD, nebulized placebo BID and tiotropium 18 μg (Spriva® HandiHaler® Boehringer Ingelheim, Ridgefield, Conn.) DPI QD, or nebulized arformoterol 15 μg BID and tiotropium 18 μg DPI QD. The nebulized drug was administered first using the PARI LC Plus® nebulizer driven by the Duraneb 3000® compressor (Pari: Pari Respiratory Equipment Inc., Midlothian, Va.) at a flow rate of 3.3 L/minute followed (within 5 minutes) by the DPI administration (HandiHaler®). The tiotropium and placebo DPI capsules were identical in size and shape but differed in color. For this reason, patients who had previously used tiotropium were excluded (see above) and the DPI capsules were dispensed and collected by an independent Study Drug Coordinator who was not otherwise involved in the study visits.

At week 0 and week 2, medical event calendars and blood samples were collected and vital signs and heart measurements analyzed. At week 0, spirometry was performed pre-morning dose, immediately (within 5 minutes) and at 30 minutes, 1, 2, 4, 6, 8, 10, and 12 hours post-first dose. After the 12-hour pulmonary function test patients self-administered the evening dose of study medication. At week 2, serial spirometry was also performed as at week 0, as well as immediately (within 5 minutes) following the evening dose (administered 12 hours after the morning dose) and 12.5, 13, 14, 16, 23, and 24 hours post-morning dose. Inspiratory capacity was evaluated pre-dose and at 2 hours post-morning dose at week 0, and pre-dose and 2, 11, 14, and 24 hours post-morning dose at week 2. All inspiratory capacity measurements were the mean of acceptable inspiratory capacity maneuvers, two of which were reproducible. Prior to an inspiratory capacity maneuver a patient had to have a stable expiratory level for about 10 breaths. Once the stable level was achieved, at the end of exhalation of a normal breath the patient was asked to make a steady and full inhalation at normal inspiratory flow rates until the lungs were completely full, and then to exhale at a normal rate.

All pulmonary function values used were the highest among the three acceptable maneuvers. The Investigator ensured that all spirometry was performed in accordance with the American Thoracic Society/European Respiratory Society Standardisation of Spirometry guidelines (see, e.g., Miller M R, Hankinson J, Brusasco V, Burgos F, Casaburi R, Coates A, et al. Standardisation of spirometry. Eur Respir J 2005; 26:319-338; herein incorporated in its entirety by reference). Centralized over-reading of spirometry and inspiratory capacity pulmonary function measures were used for quality control.

At screening, the Baseline Dyspnea Index (BDI) (see, e.g., Mahler D A, Weinberg D H, Wells C K, Feinstein A R. The measurement of dyspnea. Contents, interobserver agreement, and physiologic correlates of two new clinical indexes. Chest 1984; 85:751-758; herein incorporated in its entirety by reference) was assessed prior to the first clinic dose, and at week 2 the Transition Dyspnea Index (see id.) was evaluated before first morning dose. The baseline focal score (range 0 to 12) and the transition focal score (range −9 to 9) were the sums of the functional impairment, magnitude of task, and magnitude of effort scores (see id.). Higher scores indicate less dyspnea at baseline (BDI) or greater improvement in dyspnea from baseline (TDI).

### Statistical Methods

The study was designed to detect a mean treatment difference of time normalized FEV₁AUC over 24 hours (FEV₁AUC₀₋₂₄) (the primary endpoint) of 0.075 L with a standard deviation of 0.016 L when comparing combined therapy with mono-therapy, using a two-sided 5% significance level, following 2-weeks of dosing for the primary comparison with 80% power. All efficacy analyses were performed on the ITT population. All statistical testing was 2-tailed and conducted at the 5% significance level, unless otherwise indicated. The primary comparison is between the arformoterol plus tiotropium group versus tiotropium alone. The key secondary analysis comparison was between the arformoterol plus tiotropium group versus arformoterol alone. To control for multiple comparisons, statistical tests of mean treatment group differences were considered significant if the overall treatment effect in the model was statistically significant at the 5% level. Pulmonary function severity subgroup analysis was performed post hoc by stratifying patients according to the GOLD COPD guidelines (see, e.g., Pauwels R A, Buist A S, Calverley P M, Jenkins C R, Hurd S S. Global strategy for the diagnosis, management, and prevention of chronic obstructive pulmonary disease. NHLBI/WHO Global Initiative for Chronic Obstructive Lung Disease (GOLD) Workshop summary. Am J Respir Crit Care Med 2001; 163:1256-1276; ; herein incorporated in its entirety by reference) (<30%, ≧30% to <50%, and ≧50%, respectively). Pairwise comparisons between treatment groups were performed using least square means (LS means) from the linear model with the study baseline (or predose where applicable) as a covariate and the treatment group as a fixed effect.

Descriptive statistics were calculated by treatment for baseline characteristics and each efficacy parameter. Adverse events were summarized using counts and percentages. All adverse events were coded using MedDRA (Medical Dictionary for Regulatory Activities (see, e.g., MedDRA and MSSO. The medical dictionary for regulatory activities 2008). A COPD exacerbation was pre-defined as an increase in symptoms that necessitated any change in baseline medication other than bronchodilators (e.g. anti-inflammatory agents, antibiotics, supplemental oxygen therapy, etc.) or caused the patient to require additional medical attention (hospitalization, emergency room visit, etc.).

### Results

Of the 429 patients enrolled in this study, 235 were randomized and 234 received at least one dose of study medication (ITT population) (See FIG. 1). Demographic and baseline characteristics, including FEV₁, FVC, and inspiratory capacity values, were similar among treatment groups (See Table 1). Of the patients in the ITT population, 94.4% completed the 2-week study with similar rates of completion for all three treatment groups (See FIG. 1). The most common reason for discontinuation was the occurrence of adverse events (n=5 [2.1%]) (See FIG. 1). Approximately 97% of patients among the treatment groups were compliant with the therapies throughout the study.

TABLE 1
Demographics and baseline characteristics (ITT)

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| | n = 76 | n = 80 | n = 78 |
| Mean age, years (SD) | 61.6 (8.4) | 61.2 (9.5) | 62.2 (7.6) |
| Male, n (%) | 39 (51.3) | 43 (53.8) | 42 (53.8) |
| Race, n (%) | | | |
| Caucasian | 71 (93.4) | 74 (92.5) | 70 (89.7) |
| Black | 5 (6.6) | 6 (7.5) | 7 (9.0) |
| Other | 0 | 0 | 1 (1.3) |
| Current smoker, n (%) | 49 (64.5) | 54 (67.5) | 39 (50) |
| Pack-years smoked | | | |
| ≧15-<30 years, n (%) | 4 (5.3) | 5 (6.2) | 10 (12.8) |
| ≧30 years, n (%) | 72 (94.7) | 75 (93.8) | 68 (87.2) |
| Corticosteroid users, n | 16 (21.1) | 21 (26.3) | 16 (20.5) |
| (%)* | | | |
| MMRC Dyspnea Scale, | 2.7 (0.6) | 2.9 (0.7) | 2.9 (0.6) |
| mean (SD) | | | |
| Mean FEV₁, L (SD) | 1.37 (0.46) | 1.38 (0.46) | 1.35 (0.41) |
| Mean percent predicted | 45.4 (11.9) | 45.7 (11.5) | 44.9 (12.0) |
| FEV₁, L (SD) | | | |
| Mean FEV₁ % | 15.4 (10.0) | 15.2 (10.8) | 15.7 (13.3) |
| reversibility, (SD) | | | |
| Mean FVC, L (SD) | 2.69 (0.78) | 2.70 (0.77) | 2.60 (0.67) |
| Mean inspiratory | 2.01 (0.62) | 1.98 (0.56) | 1.92 (0.52) |
| capacity, L (SD) | | | |

*Indicates the percentage of patients that started taking inhaled or systemic corticosteroids during the screening period.

### Pulmonary Function Outcomes

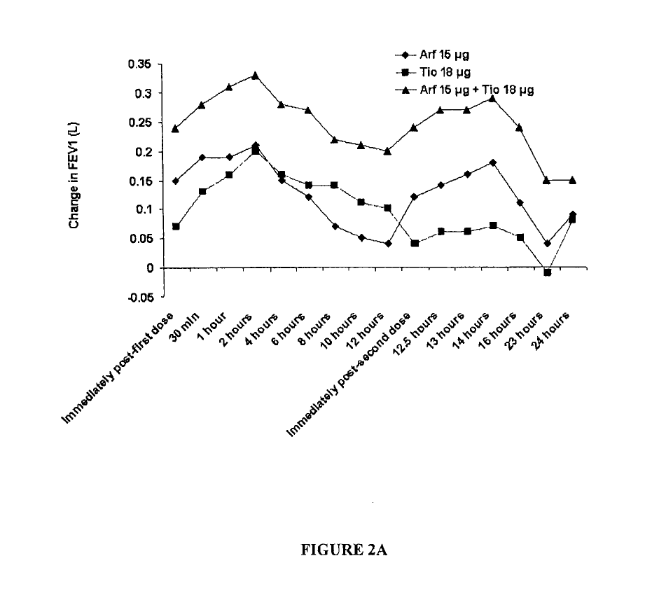

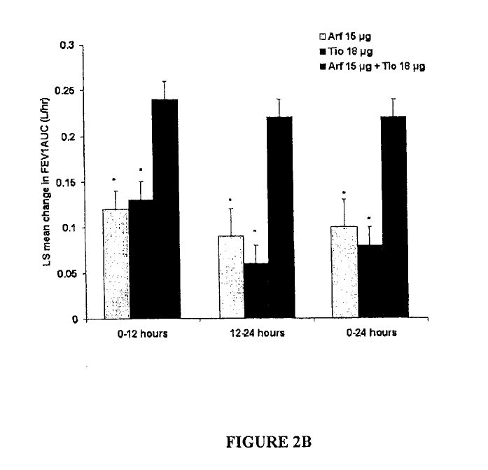

FEV₁ at each time point and time normalized FEV₁AUC₀₋₂₄, improved from baseline for all treatment groups. The two mono-therapies had comparable improvement and the combined treatment group had the greatest improvement after 2-weeks of treatment (See Table 2; FIGS. 2A and 2B). The greater change in FEV₁AUC₀₋₂₄ (the primary endpoint) for the combined therapy versus the mono-therapies was significant (p<0.001). Peak change in FEV₁, changes in trough (at end of dosing interval) FEV₁, and peak change in FVC improved significantly from baseline following all treatments (See Table 2). The mono-therapy groups improved to a similar extent and the combined therapy group had the greatest improvement. The greater increase in peak FEV₁ for combined therapy was significant versus either mono-therapies (p<0.005). The 150 mL improvement in trough FEV₁ for the combined therapy was statistically significant versus the tiotropium mono-therapy (p=0.002) and not significant versus arformoterol mono-therapy (p=0.07). The 60 mL mean improvement in peak FVC for the combined therapy was greater than that observed for either mono-therapy (tiotropium 40 mL and arformoterol 48 mL), a difference that reached statistical significance versus tiotropium (p=0.03) but not versus arformoterol (p<0.21).

The LS mean (±SE) peak improvement in FEV₁ from visit pre-dose was similar for the three treatment groups (0.19 L±0.02 for arformoterol, 0.19 L±0.02 for tiotropium and 0.22 L±0.02 for the arformoterol plus tiotropium).

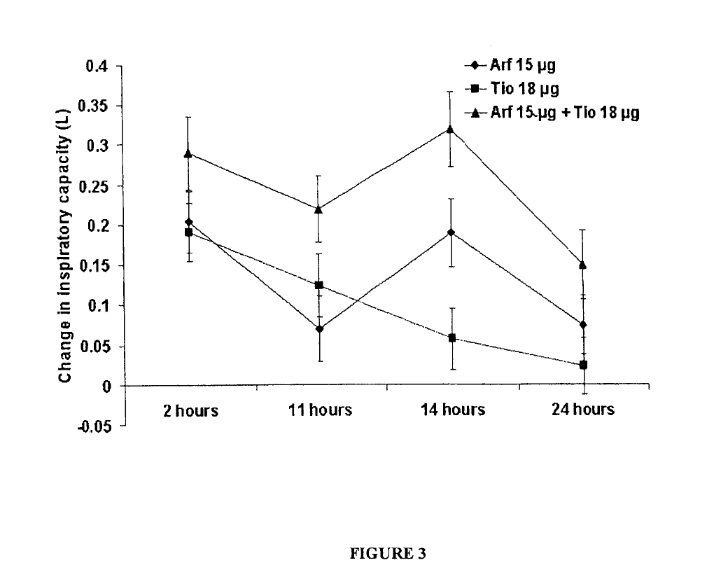

Mean (SD) inspiratory capacity improved from baseline 2-hours post-dosing for all three treatment groups, and the greatest improvement was observed for the combined therapy group (arformoterol, 0.20 L±0.32, tiotropium, 0.19 L±0.32, and arformoterol plus tiotropium, 0.29 L±0.39) (See FIG. 3). At the 24 hours time point (trough), the inspiratory capacity was significantly increased from the study baseline for the combined treatment group and approached significance for the arformoterol treatment group (See Table 2).

### Symptom Responses: Rescue Medication Use and BDII/TDI

Between screening and randomization (pre-dose week 0) about 80% of patients in all treatment groups used levalbuterol MDI as rescue medication (See Table 3). Baseline rescue use averaged approximately 3 actuations per day and about 4.5 days per week. The use of levalbuterol MDI decreased over the second week of treatment for all three treatment groups by a mean of 1.8 actuations per day for the mono-therapies and 2.5 actuations per day for the combined therapy groups. Differences for combined therapy versus mono-therapies were not statistical significance.

TABLE 2
Change in spirometry measurements from baseline at week 2

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| | n = 76 | n = 80 | n = 78 |
| Change in FEV₁AUC₀₋₂₄, (L), | 0.10 (0.21) | 0.08 (0.20) | 0.22 (0.20) |
| mean (SD) (95% C.I.) | (0.05, 0.16) | (0.04, 0.12) | (0.18, 0.27) |
| Difference between combined | 0.12 | 0.14 | |
| therapy and mono-therapies, (L), | (0.05, 0.18; | (0.08, 0.20; | |
| LS mean | p < 0.001) | p < 0.001) | |
| (95% C.I.; p-value) | | | |
| Peak change in FEV₁ over 12 | 0.27 (0.21) | 0.27 (0.23) | 0.38 (0.22) |
| hours, (L), mean (SD) | (0.22, 0.32) | (0.21, 0.32) | (0.33, 0.43) |
| (95% C.I.) | | | |
| Difference between combined | 0.11 | 0.11 | |
| therapy and mono- therapies, | (0.03, 0.18; | (0.04, 0.19; | |
| (L), LS mean | p = 0.004) | p = 0.002) | |
| (95% C.I.; p-value) | | | |
| Change in trough FEV₁ (L), mean | 0.09 (0.23) | 0.08 (0.21) | 0.15 (0.22) |
| (SD) | (0.03, 0.14) | (0.03, 0.13) | (0.10, 0.21) |
| (95% C.I.)* | | | |
| Difference between combined | 0.07 | 0.07 | |
| therapy and mono- therapies, | (−0.01, 0.14; | (0.0, 0.14; | |
| (L), LS mean (95% C.I.; p- | p = 0.07) | p = 0.05) | |
| value) | | | |
| Peak change in FVC over 12 | 0.48 (0.37) | 0.40 (0.34) | 0.60 (0.43) |
| lours (L), mean (SD) | (0.39, 0.57) | (0.32, 0.48) | (0.50, 0.70) |
| (95% C.I.) | | | |
| Difference between combined | 0.12 | 0.20 | |
| therapy and mono- therapies, | (−0.01, 0.25; | (0.08, 0.33; | |
| (L), LS mean (95% C.I.; p- | p = 0.07) | p = 0.002) | |
| value) | | | |
| Change in trough inspiratory | 0.07 (0.30) | 0.02 (0.29) | 0.15 (0.36) |
| capacity (L), mean (SD)* | (0.00, 0.15) | (−0.05, 0.09) | (0.07, 0.24) |
| 95% C.I. | | | |
| Difference in trough FEV₁ | 0.07 | 0.12 | |
| between combined therapy and | (−0.04, 0.18; | (0.02, 0.23; | |
| mono- therapies, (L), LS mean | p = 0.21) | p = 0.03) | |
| (95% C.I.; p-value) | | | |

*Trough is defined as the given pulmonary function variable measured at the 24 hour time point after morning dose.

TABLE 3
Daily rescue medication (levalbuterol) use

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| | n = 76 | n = 80 | n = 78 |
| Baseline (prior to first | | | |
| dose week 0) | | | |
| Used levalbuterol, n (%) | 61 (80.3) | 64 (80.0) | 65 (83.3) |
| Number of actuations per | 3.2 (3.2) | 2.8 (2.8) | 3.1 (2.7) |
| day, mean (SD) | | | |
| Number of days per | 4.4 (2.8) | 4.3 (2.9) | 4.6 (2.8) |
| week, mean (SD) | | | |
| Week 2 | | | |
| (change from baseline) | | | |
| Used levalbuterol, n (%) | 40 (52.6) | 38 (47.5) | 26 (33.3) |
| Number of actuations per | −1.8 (2.2) | −1.8 (2.8) | −2.5 (2.3) |
| day, mean (SD) | | | |
| Number of days per | −2.1 (2.6) | −2.2 (2.7) | −3.3 (3.0) |
| week, mean (SD) | | | |

TABLE 4
Baseline Dyspnea (BDI)/Transitional Dyspnea
Index (TDI) at week 2 for the ITT population+

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| | n = 76 | n = 80 | n = 78 |
| BDI, mean (SD) | 5.8 (2.0) | 5.8 (1.9) | 5.5 (2.1) |
| TDI, mean (SD) | 2.3 (2.4) | 1.8 (2.8) | 3.1 (2.4) |
| Difference between | 0.9 | 1.3 | |
| combined therapy and | (0.03, 1.7) | (0.5, 2.2) | |
| mono- therapies, (L) | | | |
| LS mean (95% C.I.) | | | |
| Patients with | 50 (66.7) | 44 (57.1) | 60 (77.9) |
| change ≧1 | | | |
| unit, n (%) | | | |

Dyspnea, as measured by TDI, improved from baseline for all three treatment groups and to a significantly greater extent for the combined treatment group (See Table 4). The majority of patients in the three treatment groups had an improvement in TDI of ≧1 unit, the minimal clinically important difference. The combined therapy group had a greater proportion of patients with ≧1 unit improvement in TDI compared with the other two therapy groups, and this difference was significant between combined and tiotropium therapies.

### Pulmonary Function and Disease Symptom Outcomes Stratified by Patient's Baseline Lung Function Severity

Pulmonary results stratified by baseline disease severity (pre-dose FEV₁<50% predicted or ≧50% predicted), demonstrated that patients with lower baseline lung function had greater improvement in all pulmonary lung function measures than patients with higher baseline lung function (See Tables 5, 6, and 7). The greater improvement in pulmonary function measures for those patients with more compromised baseline lung function (<50% FEV₁ predicted) was evident for both absolute (L) and relative (percentage) improvements. Patients with <50% FEV₁ predicted demonstrated significant improvement for all five forced expiratory measures evaluated for both the mono-therapies and combined therapy groups. In contrast, patients with >50% FEV₁ predicted had no significant improvement in trough FEV₁ for any therapy group, and FEV₁AUC₀₋₂₄ only demonstrated improvement for the combined therapy group.

The use of rescue medications decreased for both disease severity groups (See Table 8). Both subsets of patients had improved dyspnea following any of the three therapies (See Table 8). Patients with <50% predicted FEV₁ at baseline treated with the combined therapy had significantly greater improvement in TDI (3.5 units) than those treated with either arformoterol (2.3 units) or tiotropium (1.6 units) (See Table 9).

TABLE 5
Baseline pulmonary function outcomes stratified
by patient's baseline percent predicted FEV₁

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| Percent predicted | n = 47 | n = 51 | n = 48 |
| FEV₁ <50% | | | |
| FEV₁, (L), mean (SD) | 1.19 (0.34) | 1.21 (0.35) | 1.13 (0.27) |
| FVC, (L), mean (SD) | 2.61 (0.77) | 2.63 (0.78) | 2.42 (0.57) |
| Inspiratory capacity, (L), | 1.89 (0.55) | 1.96 (0.57) | 1.83 (0.44) |
| mean (SD) | | | |
| Percent predicted | n = 29 | n = 28 | n = 30 |
| FEV₁ ≧50% | | | |
| FEV₁, (L), mean (SD) | 1.66 (0.47) | 1.68 (0.49) | 1.70 (0.35) |
| FVC, (L), mean (SD) | 2.82 (0.78) | 2.84 (0.73) | 2.89 (0.74) |
| Inspiratory capacity, (L), | 2.20 (0.68) | 2.00 (0.55) | 2.08 (0.60) |
| mean (SD) | | | |

TABLE 6
Change in FEV₁AUC₀₋₂₄ from study baseline (pre-dose week 0) at
week 2 stratified by patient's baseline percent predicted FEV₁

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| Percent predicted | n = 45 | n = 48 | n = 44 |
| FEV₁ <50% | | | |
| Change in | 0.15 (0.22) | 0.10 (0.22) | 0.25 (0.21) |
| FEV₁AUC₀₋₂₄, | (0.08, 0.21) | (0.03, 0.16) | (0.19, 0.32) |
| (L), mean (SD) | | | |
| (95% C.I.) | | | |
| Difference between | 0.11 | 0.16 | |
| combined therapy | (0.02, 0.20; | (0.07, 0.25; | |
| and mono-therapies, | p = 0.02) | p < 0.001) | |
| (L), LS mean | | | |
| (95% C.I.; p-value) | | | |
| Percent predicted | n = 26 | n = 27 | n = 28 |
| FEV₁ ≧50% | | | |
| Change in | 0.03 (0.19) | 0.05 (0.14) | 0.17 (0.16) |
| FEV₁AUC₀₋₂₄, | (−0.44, 0.11) | (−0.01, 0.11) | (0.11, 0.23) |
| (L), mean (SD) | | | |
| (95% C.I.) | | | |
| Difference between | 0.14 | 0.12 | |
| combined therapy | (0.04, 0.24; | (0.04, 0.21; | |
| and mono-therapies, | p = 0.005) | p = 0.004) | |
| (L), LS mean | | | |
| (95% C.I.; p-value) | | | |

TABLE 7
Peak change in FEV₁ over 12 hours, change in FEV₁ at 24 hours
post-dose (trough), peak change in FVC over 12 hours and inspiratory
capacity at week 2 from study baseline (pre-dose week 1) stratified
by patient's baseline percent predicted FEV₁

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| Peak Change in FEV₁ over | | | |
| 12 hours | | | |
| Baseline Percent | n = 45 | n = 48 | n = 46 |
| predicted FEV₁ <50% | | | |
| (L), mean (SD) | 0.31 (0.23) | 0.29 (0.26) | 0.41 (0.23) |
| (95% C.I.) | (0.24, 0.38) | (0.21, 0.36) | (0.34, 0.48) |
| Difference between | 0.11 | 0.14 | |
| combined therapy and | (0.01, 0.21; | (0.03, 0.24; | |
| mono- therapies, (L), | p = 0.03) | p = 0.01) | |
| LS mean | | | |
| (95% C.I.; p-value) | | | |
| Baseline Percent | n = 26 | n = 27 | n = 28 |
| predicted FEV₁ ≧50% | | | |
| (L), mean (SD) | 0.22 (0.17) | 0.23 (0.16) | 0.33 (0.20) |
| (95% C.I.) | (0.15, 0.28) | (0.17, 0.30) | (0.25, 0.40) |
| Difference between | 0.10 | 0.09 | |
| combined therapy and | (0.01, 0.20; | (−0.001, 0.19; | |
| mono- therapies, (L), | p = 0.04) | p = 0.05) | |
| LS mean | | | |
| (95% C.I.; p-value) | | | |
| Trough FEV₁ | | | |
| Baseline Percent | n = 44 | n = 46 | n = 46 |
| predicted FEV₁ <50% | | | |
| (L), mean (SD) | 0.13 (0.23) | 0.11 (0.22) | 0.21 (0.23) |
| (95% C.I.) | (0.06, 0.20) | (0.05, 0.18) | (0.14, 0.28) |
| Difference between | 0.08 | 0.10 | |
| combined therapy and | (−0.01, 0.18; | (0.01, 0.19; | |
| mono- therapies, (L), | p = 0.09) | p = 0.04) | |
| LS mean | | | |
| (95% C.I.; p-value) | | | |
| Baseline Percent | n = 25 | n = 27 | n = 28 |
| predicted FEV₁ ≧50% | | | |
| (L), mean (SD) | 0.02 (0.21) | 0.03 (0.17) | 0.06 (0.19) |
| (95% C.I.) | (−0.07, 0.11) | (−0.04, 0.10) | (−0.01, 0.14) |
| Difference between | 0.05 | 0.04 | |
| combined therapy and | (−0.06, 0.16; | (−0.06, 0.14; | |
| mono- therapies, (L) LS | p = 0.37) | p = 0.43) | |
| mean | | | |
| (95% C.I.; p-value) | | | |
| Peak change in FVC over 12 | | | |
| hours | | | |
| Baseline Percent | n = 45 | n = 48 | n = 46 |
| predicted FEV₁ <50% | | | |
| (L), mean (SD) | 0.56 (0.41) | 0.43 (0.37) | 0.71 (0.44) |
| (95% C.I.) | (0.44, 0.68) | (0.32, 0.54) | (0.58, 0.84) |
| Difference between | 0.15 | 0.28 | |
| combined therapy and | (−0.03, 0.33; | (0.11, 0.45; | |
| mono- therapies, (L), | p = 0.10) | p = 0.001) | |
| LS mean | | | |
| (95% C.I.; p-value) | | | |
| Baseline Percent | n = 26 | n = 27 | n = 28 |
| predicted FEV₁ ≧50% | | | |
| (L), mean (SD) | 0.34 (0.25) | 0.34 (0.28) | 0.43 (0.35) |
| (95% C.I.) | (0.24, 0.44) | (0.23, 0.45) | (0.29, 0.56) |
| Difference between | 0.08 | 0.08 | |
| combined therapy and | (−0.08, 0.24; | (−0.08, 0.25; | |
| mono- therapies, (L), | p = 0.33) | p = 0.32) | |
| LS mean | | | |
| (95% C.I.; p-value) | | | |
| Change in Inspiratory | | | |
| Capacity | | | |
| Baseline Percent | n = 42 | n = 43 | n = 43 |
| predicted FEV₁ <50% | | | |
| (L), mean (SD) | 0.12 (0.31) | 0.02 (0.27) | 0.21 (0.38) |
| (95% C.I.) | | | |
| | (0.02, 0.22) | (−0.07, 0.10) | (0.09, 0.32) |
| Difference between | 0.08 | 0.17 | |
| combined therapy and | (−0.07, 0.23; | (0.03, 0.31; | |
| mono- therapies, (L) LS | p = 0.27) | p = 0.02) | |
| mean | | | |
| (95% C.I.; p-value) | | | |
| Baseline Percent | n = 24 | n = 25 | n = 27 |
| predicted FEV₁ ≧50% | | | |
| (L), mean (SD) | −0.01 (0.27) | 0.04 (0.33) | 0.06 (0.31) |
| (95% C.I.) | (−0.12, 0.11) | (−0.10, 0.18) | (−0.06, 0.18) |
| Difference between | 0.06 | 0.02 | |
| combined therapy and | (−0.10, 0.22; | (−0.15, 0.20; | |
| mono-therapies, (L) LS | p = 0.45) | p = 0.79) | |
| mean | | | |
| (95% C.I.; p-value) | | | |

TABLE 8
Daily rescue medication (levalbuterol MDI) use

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| <50% percent | n = 47 | n = 51 | n = 48 |
| predicted FEV₁ | | | |
| Baseline (prior to first | | | |
| dose week 0) | | | |
| Used levalbuterol, n (%) | 41 (87.2) | 41 (80.4) | 40 (83.3) |
| Number of actuations per | 3.6 (3.2) | 3.1 (3.1) | 3.4 (2.9) |
| day, mean (SD) | | | |
| Changes in levalbuterol | | | |
| MDI use at week 2 | | | |
| Used levalbuterol, n (%) | 45 (95.7) | 48 (94.1) | 47 (97.9) |
| Number of actuations per | −2.1 (2.3) | −1.8 (3.3) | −2.8 (2.5) |
| day, mean (SD) | | | |
| ≧50% percent | n = 29 | n = 28 | n = 30 |
| predicted FEV₁ | | | |
| Baseline (prior to first | | | |
| dose week 0) | | | |
| Used levalbuterol, n (%) | 20 (69.0) | 22 (78.6) | 25 (83.3 |
| Number of actuations per | 2.6 (3.1) | 2.3 (2.0) | 2.6 (2.4) |
| day, mean (SD) | | | |
| Changes in levalbuterol | | | |
| MDI use at week 2 | | | |
| Used levalbuterol, n (%) | 26 (90) | 27 (96.4) | 28 (93.3) |
| Number of actuations per | −1.3 (2.0) | −1.9 (1.8) | −1.9 (1.7) |
| day, mean (SD) | | | |

TABLE 9
Baseline Dyspnea (BDI)/Transitional
Dyspnea Index (TDI) at week 2

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| BDI, mean (SD) | | | |
| Baseline Percent | 5.6 (1.8) | 5.8 (1.8) | 5.3 (2.1) |
| predicted FEV₁ <50% | | | |
| Baseline Percent | 6.2 (2.4) | 5.7 (2.1) | 5.8 (2.0) |
| predicted FEV₁ ≧50% | | | |
| TDI | | | |
| Baseline Percent | n = 47 | n = 48 | n = 47 |
| predicted FEV₁ <50% | | | |
| mean, (SD) | 2.3 (2.3) | 1.6 (3.0) | 3.5 (2.3) |
| (95% C.I.) | (1.6, 2.9) | (0.7, 2.4) | (2.9, 4.2) |
| Difference between | 1.3 | 2.0 | |
| combined therapy and | (0.2, 2.3) | (0.9, 3.0) | |
| mono- therapies, LS | | | |
| mean | | | |
| (95% C.I.) | | | |
| Patients with change | 34 (72.3) | 25 (52.1) | 40 (85.1) |
| of ≧1 unit, n (%) | | | |
| Baseline Percent | n = 28 | n = 28 | n = 30 |
| predicted FEV₁ ≧50% | | | |
| mean, (SD) | 2.3 (2.6) | 2.3 (2.5) | 2.5 (2.6) |
| (95% C.I.) | (1.3, 3.3) | (1.3, 3.2) | (1.5, 3.5) |
| Difference between | 0.2 | 0.3 | |
| combined therapy and | (−1.2, 1.5) | (−1.1, 1.6) | |
| mono- therapies, LS | | | |
| mean | | | |
| (95% C.I.) | | | |
| Patients with change | 17 (57.1) | 19 (67.9) | 20 (66.7) |
| of ≧1 unit, % | | | |

### Safety

Adverse events were infrequent with similar occurrence among the three treatment groups (See Table 10). Both COPD exacerbations and cardiovascular adverse events were observed in only a small proportion of patients (between 0 to 3.9%). Only one patient (arformoterol 15 μg) reported a serious adverse event (small intestinal obstruction).

TABLE 10
Adverse events

| | | | Arformoterol |
|---|---|---|---|
| | | | 15 μg BID |
| | Arformoterol | Tiotropium | plus Tiotropium |
| | 15 ug BID | 18 μg QD | 18 μg QD |
| | n = 76 | n = 80 | n = 78 |
| Any adverse event, n (%) | 19 (25.0) | 22 (27.5) | 24 (30.8) |
| COPD exacerbations | 3 (3.9) | 0 | 0 |
| Overall | 2 (2.6) | 1 (1.3) | 0 |
| cardiovascular adverse | | | |
| events, n (%) | | | |
| Discontinued | 2 (2.6) | 1 (1.3) | 2 (2.6) |
| due to adverse | | | |
| events, n (%) | | | |
| Serious adverse | 1 (1.3) | 0 | 0 |
| events, n (%) | | | |

### Further Discussion

This study investigated the efficacy and safety of the combination of two long-acting bronchodilators: arformoterol administered via nebulizer and tiotropium administered as a DPI. In particular, it compared efficacy between the two mono-therapies and evaluated whether the combinated use of these drugs resulted in greater pulmonary improvement than either single-agent alone.

All three therapies demonstrated clinically meaningful improvement in pulmonary function from baseline after 2-weeks of treatment. However, the combined use of arformoterol and tiotropium was associated with significantly larger increases in time normalized FEV₁ over a 24-hour period and peak change in FEV₁ than either arformoterol or tiotropium mono-therapies. Trough FEV₁ (24 hours post-dose at week 2), another efficacy measure for a maintenance bronchodilator, improved for all three treatment groups, indicating that bronchodilation was maintained throughout the dosing interval. The combination therapy resulted in a 70 mL greater improvement in trough FEV₁ than either mono-therapy.

In this study, the improvement in FEV₁ after arformoterol dosing differed between the morning and evening dose. The mean FEV₁ improvement 2-hours after the morning dose and evening dose was approximately 213 mL and 182 mL, respectively. This temporal difference in response has been reported for racemic formoterol administered BID and was suggested to reflect circadian changes in the activity of the adrenergic system and vagal system. The adrenergic system is most prominent during the day and the parasympathetic system activity increases during the night. The relative reduction in the effect of tiotropium between 12 and 23 hours may also result from this circadian nocturnal drop in airway function and the waning effect of tiotropium that dosed once daily in the morning.

Inspiratory capacity and dyspnea, both reflections of hyperinflation, improved in this study after dosing for all three treatments and to a greater extent in the combined treatment group. Similar to the findings for trough FEV₁, the fact that trough inspiratory capacity (at the 24 hour time point at week 2) was greater than baseline indicates that the effect of the three therapies on this outcome persisted for 24 hours. In contrast to prior reports that examined the combination of tiotropium and racemic formoterol (see, e.g., O'Donohue W J, Jr. Guidelines for the use of nebulizers in the home and at domiciliary sites. Report of a consensus conference. National Association for Medical Direction of Respiratory Care (NAMDRC) Consensus Group. Chest 1996; 109:814-820; van Noord J A, Aumann J L, Janssens E, Verhaert J, Smeets J J, Mueller A, et al. Effects of Tiotropium With and Without Formoterol on Airflow Obstruction and Resting Hyperinflation in Patients With COPD. Chest 2006; 129:509-517.), this study found that the combined effect of tiotropium and arformoterol on trough inspiratory capacity was significantly greater than that of tiotropium alone. Dyspnea improved by more than 1 unit (the MCID) (see Witek T J, Jr., Mahler D A. Minimal important difference of the transition dyspnoea index in a multinational clinical trial. Eur Respir J 2003; 21:267-272) for all three therapies and greatest (mean TDI; +3.1 units) for the combined therapy. Rescue short-acting β₂-agonist use decreased with all three therapies and again to a slightly greater extent with combination therapy than either mono-therapy.

In this study, stratified analysis of the response of patients based on baseline GOLD guideline classification of disease severity (e.g. very severe and severe: <50% predicted FEV₁; and moderate: ≧50% predicted FEV₁) (see, e.g., Rabe K F, Hurd S, Anzueto A, Barnes P J, Buist S A, Calverley P, et al. Global Strategy for the Diagnosis, Management, and Prevention of Chronic Obstructive Pulmonary Disease: GOLD Executive Summary Am J Respir Crit Care Med 2007; 176:532-555.) demonstrated that patients with more severe COPD had greater airway improvement than those with moderate COPD. Pre-dose (trough) and post-dose FEV₁ values increased more for patients with more severe COPD compared with those with moderate disease. Moreover, trough inspiratory capacity increased only for patients with more severe disease. Improvements in dyspnea (TDI), in contrast, were similar between disease severity groups. These findings suggest that disease severity influences the degree of bronchodilator improvements in forced expiratory maneuvers and inspiratory capacity. These findings are in contrast to a prior study that found that patients with very severe COPD (GOLD stage III and IV) had less responsiveness to large doses of the short-acting β₂-agonist racemic albuterol plus ipratropium bromide than patients with moderate COPD (see, e.g., Tashkin D P, Celli B, Decramer M, Liu D, Burkhart D, Cassino C, et al. Bronchodilator responsiveness in patients with COPD. Eur Respir J 2008; 31:742-750.).

In this study, the administration of tiotropium QD plus arformoterol BID resulted in significantly superior bronchodilation to either agent alone as well as significantly greater improvement in symptom relief. COPD subjects with a more severe degree of airway function compromise had greater improvement in lung function and symptoms than those with moderate impairment.

### Example 2

### Example of Preparation

Various embodiments of the compositions of the present inventions can be prepared by a person of skill in the art as follows. For example, in one method, a solution of NaCl can be prepared with concentration approximately 9 g/l. To this can be added tiotropium bromide to a concentration as desired, but typically about 4 to about 10 μg/ml, for a 2 ml total volume, and arformoterol, again to the concentration desired but typically about 3.5 to about 8 μg/ml, for a 2 ml total volume. In various embodiments, HCl is then added to give a final pH of about 4.0. In various embodiments, HCl is then added to give a final pH of about 3.0. This composition can be filled into ampoules (e.g., by blow-fill-seal techniques) to yield ampoules with the required extractable volume of composition.

All literature and similar material cited in this application, including, but not limited to, patents, patent applications, articles, books, treatises, and web pages, regardless of the format of such literature and similar materials, are expressly incorporated by reference in their entirety for all purposes. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the present inventions have been described in conjunction with various embodiments and examples, it is not intended that the present inventions be limited to such embodiments or examples. On the contrary, the present inventions encompass various alternatives, modifications, and equivalents, as will be appreciated by those of skill in the art. Therefore, all embodiments that come within the scope and spirit of the present inventions and equivalents thereto are claimed.

What is claimed is:

1. A pharmaceutical composition comprising tiotropium, or a pharmaceutically acceptable salt thereof, in an amount between 5 μg to 30 μg based on tiotropium, and arformoterol, or a pharmaceutically acceptable salt thereof, together in water or a water-ethanol mixture, wherein said tiotropium and said arformoterol, or their pharmaceutically acceptable salts, are the only active ingredients in the composition.

2. A liquid, propellant-free pharmaceutical composition comprising:
(a) tiotropium, or a pharmaceutically acceptable salt or hydrate thereof, in an amount between about 5 μg to about 30 μg based on tiotropium; and
(b) a formoterol component comprising arformoterol, or a pharmaceutically acceptable salt, or hydrate thereof, in an amount between about 6 μg to about 40 μg based on arformoterol;
wherein the tiotropium and formoterol component are dissolved together in a liquid carrier, and wherein the formoterol component comprises less than about 10% by weight of stereoisomers of formoterol other than arformoterol;
and wherein said tiotriopium and said formoterol, or their pharmaceutically acceptable salts, are the only active ingredients in the composition.

3. The pharmaceutical composition of claim 2, wherein the liquid composition has a pH in the range between 3.0 to 5.5.

4. The pharmaceutical composition of claim 3, wherein the liquid composition has a pH in the range between 3.0 to 4.0.

5. The pharmaceutical composition of claim 2, wherein the formoterol component comprises greater than 99% by weight of arformoterol and less than 1% by weight of stereoisomers of formoterol other than arformoterol.

6. The pharmaceutical composition of claim 2, wherein the carrier comprises water.

7. The pharmaceutical composition of claim 6, wherein the carrier is a water/ethanol mixture.

8. The pharmaceutical composition of claim 2, wherein the tiotropium, or a pharmaceutically acceptable salt or hydrate thereof, is present in an amount between 5 μg to 15 μg based on tiotropium.

9. The pharmaceutical composition of claim 2, wherein the arformoterol portion of the formoterol component, or a pharmaceutically acceptable salt or hydrate thereof, is present in an amount between 6 μg to 30 μg based on arformoterol.

10. The pharmaceutical composition of claim 2, wherein:
(a) the tiotropium, or a pharmaceutically acceptable salt or hydrate thereof, is present in an amount between 5 μg to 15 μg based on tiotropium; and;
(b) the arformoterol portion of the formoterol component, or a pharmaceutically acceptable salt or hydrate thereof, is present in an amount between 12 μg to 30 μg based on arformoterol.

11. The pharmaceutical composition of claim 2, wherein the liquid, propellant-free pharmaceutical composition is provided with a total liquid volume between 1 ml to 3 ml.

12. The pharmaceutical composition of claim 2, wherein the liquid, propellant-free pharmaceutical composition is provided with a total liquid volume less than 2 ml.

13. The pharmaceutical composition of claim 2, wherein the pharmaceutical composition is provided in an ampoule as a liquid for nebulization.

14. A method of treating conditions associated with reversible obstruction of the airways comprising the administration of a pharmaceutical composition of claim 2, wherein the method comprises administering a total per day dose of arformoterol between 6 to 150 μg and a total per day dose of tiotropium between 8 to 150 μg.

15. The method of treating conditions associated with reversible obstruction of the airways of claim 14, wherein the method comprises administering a total per day dose of arformoterol between 15 to 45 μg and a total per day dose of tiotropium between 18 to 54 μg.

16. The method of treating conditions associated with reversible obstruction of the airways of claim 14, wherein the conditions associated with reversible obstruction of the airways comprises COPD.

17. The method of treating conditions associated with reversible obstruction of the airways of claim 14, wherein the conditions associated with reversible obstruction of the airways comprises asthma.

18. The method of treating conditions associated with reversible obstruction of the airways of claim 14, wherein the method comprises administration of the pharmaceutical composition by nebulization.

19. A method of preventing bronchoconstriction or inducing bronchodilation in a mammal by administering a pharmaceutical composition of claim 2.

20. The method of claim 19, wherein the method comprises administering a total per day dose of arformoterol between 6 to 150 μg and a total per day dose of tiotropium between 8 to 150 μg.

21. The method of claim 19, wherein the method comprises administering a total per day dose of arformoterol between 15 to 45 μg and a total per day dose of tiotropium between 18 to 54 μg.

22. The method of claim 19, wherein the method comprises administration of the pharmaceutical composition by nebulization.

23. The method of claim 22, wherein the pharmaceutical composition is provided as propellant-free liquid composition with a total liquid volume between 1 ml to 3 ml.

24. The method of claim 22, wherein the pharmaceutical composition is provided as propellant-free liquid composition with a total liquid volume of less than 2 ml.
